(19) United States
(12) Patent Application Publication
(10) Pub. No.: US 2024/0050484 A1
(43) Pub. Date: Feb. 15, 2024

(54) CELL GEL PREPARATION FOR REDUCING SHEAR DAMAGE ON CELLS AND METHOD FOR REDUCING SHEAR DAMAGE ON CELLS

(71) Applicant: Tsinghua University, Beijing (CN)

(72) Inventors: Dongsheng Liu, Beijing (CN); Jiakuo Yu, Beijing (CN); Bo Yang, Beijing (CN); Xin Yan, Beijing (CN); Yujie Li, Beijing (CN); Bini Zhou, Beijing (CN); Xin Li, Beijing (CN)

(21) Appl. No.: 18/286,080

(22) PCT Filed: Oct. 8, 2021
(86) PCT No.: PCT/CN2021/122674

§ 371 Date: Oct. 6, 2023

(30) Foreign Application Priority Data
Apr. 9, 2021 (CN) 202110386052.7

(51) Int. Cl. A61K 35/28; A61K 9/06; A61K 47/26; A61K 9/00; A61P 19/02

(52) U.S. Cl. CPC A61K 35/28; A61K 9/06; A61K 47/26; A61K 9/0019; A61P 19/02

(57) ABSTRACT

The present invention relates to a cell gel preparation, which is used for treating and/or preventing acute or chronic osteoarthritis and/or symptoms thereof by means of intra-articular injection, wherein the preparation comprises a nucleic acid hydrogel loading mesenchymal stem cells. The present invention further relates to a method for treating or preventing acute and chronic osteoarthrosis and associated symptoms caused by inflammation (especially osteoarticular pain and activity or function loss) by means of using the cell gel preparation, and a method for reducing the shear damage on cells in a shear environment by means of using the nucleic acid hydrogel.

### TECHNICAL FIELD

The present invention relates to a cell gel preparation for treating and/or preventing acute or chronic osteoarthritis and/or symptoms thereof by means of intra-articular injection, comprising a nucleic acid hydrogel loaded with mesenchymal stem cells; a method for treating or preventing acute and chronic osteoarthrosis and associated inflammation-derived symptoms (especially osteoarticular pain and activity or function loss) using the cell gel preparation; and a method for reducing the shear damage on cells in a shear environment using the nucleic acid hydrogel.

### BACKGROUND ART

Osteoarthritis (OA) is a chronic disease characterized by degenerative changes of articular cartilage and secondary bone hyperplasia. The regenerative capacity of chondrocytes is limited, so the degenerative changes of cartilage are irreversible. Mesenchymal stem cells (MSCs) have been confirmed to have a certain degree of efficacy in repairing cartilage and delaying the degenerative changes, and are of great significance in the treatment of osteoarthritis and cartilage defects.

Currently, cell therapy is one of the treatment methods with good clinical application prospects, and in the traditional cell therapy, an aqueous solution is used as the cell delivery vehicle. For example, in the traditional cell therapy of osteoarthritis, a cell suspension is injected into the joint cavity via a syringe to exert its biotherapeutic effect. However, in the process of delivering cells in an aqueous solution, a large number of cells will die within a few hours to a few days after injection. Currently, there are three widely accepted mechanisms for the explanation of the massive cell death during the transfer process: cells die from the shear force on them during the injection process; the lack of growth factors results in the death of anchorage-dependent cells; and the injected foreign cells die from the lack of the nutritional support from the host tissue as they are difficult to access the blood vessels in the host tissue. Among them, mechanical shear force during the injection process is an important cause of cell death. When cells are injected in a Newtonian fluid such as normal saline, they are subjected to shear and tensile forces within the syringe. This is due to the flow resistance at the interface between the inner wall of the syringe and the fluid, such that the flow velocity at the center of the syringe is higher than that near the inner wall. In addition, the diameter of the needle used for injection is typically smaller than that of the syringe, which results in a dramatic increase in the tensile force at the syringe/needle interface. The uneven distribution of these forces will subject the cells to extreme shear stress, resulting in cell membrane rupture and rapid death of some necrotic cells, and may trigger an apoptotic process, resulting in further death of the cells which are injected into the host. Moreover, the presence of shear force during the injection process may also affect the gene expression and phenotype of cells, etc., and especially has an important effect on the differentiation of stem cells.

A shear-thinning biomaterial can liquefy to form a lubricating layer on the inner wall surface of the syringe due to the presence of shear stress, thereby reducing the flow resistance of the overall material, and then during the injection process, the material has a similar flow velocity at the center and edge of the syringe, forming a plug flow, thereby reducing the shear stress. Therefore, it can provide mechanical protection for the cells during the injection process, and reduce the shear force on the cells, thereby improving the cell survival rate and reducing the effect of shear force on the gene expression in the cells, etc. Such biomaterials include natural macromolecular and supramolecular hydrogels prepared based on hyaluronic acid, sodium alginate, etc., polypeptide-based supramolecular hydrogels, and the like. Although these materials have certain shear thinning properties, they have shortcomings such as hidden dangers in biological safety and lack of structural permeability, which hinder their applications in further cell culture and tissue regeneration.

In addition to cell damage caused by shear during the injection process, the cells injected into the joint are also damaged due to such shear environment as friction within the joint, thereby affecting their efficacy. Thus, there remains a need for further improved shear thinning properties to further reduce cell damage in a shear environment.

As for the problems that the cell survival rate and therapeutic effect are greatly reduced due to cell damage caused by shear during the cell injection process by using an aqueous solution in the traditional cell therapy, and the existing shear-thinning materials have hidden dangers in biological safety and are lack of structural permeability, there are an urgent need for a method that can further reduce the shear damage on cells in the treatment of osteoarthritis, as well as a cell preparation that reduces the shear damage on cells and has good biocompatibility and other desirable properties required for the treatment of osteoarthritis at the same time.

### REFERENCES

1. Piuzzi, N. S.; Ng, M.; Chughtai, M.; Khlopas, A.; Ramkumar, P. N.; Harwin, S. F.; Mont, M. A.; Bauer, T. W.; Muschler, G. F., Accelerated Growth of Cellular Therapy Trials in Musculoskeletal Disorders: An Analysis of the NIH Clinical Trials Data Bank. Orthopedics 2019, 42 (2), e144-e150.

2. Jones, I. A.; Togashi, R.; Wilson, M. L.; Heckmann, N.; Vangsness, C. T., Jr., Intra-articular treatment options for knee osteoarthritis. Nat Rev Rheumatol 2019, 15 (2), 77-90.

3. Ng, J.; Little, C. B.; Woods, S.; Whittle, S.; Lee, F. Y; Gronthos, S.; Mukherjee, S.; Hunter, D. J.; Worthley, D. L., Stem cell directed therapies for osteoarthritis: The promise and the practice: Concise review. Stem Cells 2019.

4. Mitrousis, N.; Fokina, A. & Shoichet, M.S. Biomaterials for cell transplantation. Nat Rev Mater 2018, 3, 441-456.

5. Yan, C.; Mackay M. E.; Czymmek K.; Nagarkar R. P.; Schneider J. P.; and Pochan D. J., Injectable Solid peptide hydrogel as a cell carrier: effects of shear flow on hydrogels and cell payload. Langmuir 2012 28 (14), 6076-6087

### SUMMARY OF THE INVENTION

The present inventors have found that when a specific nucleic acid-based supramolecular hydrogel is used as the cell delivery vehicle, the nucleic acid hydrogel can provide almost 100% protection for the therapeutic cells, and the cell survival rate can be as high as 99% or more during the cell injection process and when simulating the shear environment within the joint. The present inventors have found that, since the nucleic acid hydrogel has a unique three-dimensional network structure and excellent shear thinning properties, it can significantly reduce the shear damage on cells, provide anti-shear protection for cells, and improve the cell survival rate in a shear environment, thus improving the efficacy of cell therapy. On the other hand, compared with natural macromolecular and supramolecular hydrogels prepared based on hyaluronic acid, sodium alginate, etc., the nucleic acid-based supramolecular hydrogel of the present invention has excellent properties suitable for cartilage tissue engineering, such as rapid gelation, good biocompatibility, and good permeability, and is thus suitable for forming a cell gel preparation for the treatment of osteoarthritis.

Based on the above findings, the present invention has been achieved.

Specifically, the present invention relates to a cell gel preparation for treating and/or preventing acute or chronic osteoarthritis and/or symptoms thereof by means of intra-articular injection, comprising a nucleic acid hydrogel loaded with mesenchymal stem cells; a method for treating or preventing acute and chronic osteoarthrosis and associated inflammation-derived symptoms (especially osteoarticular pain and activity or function loss) using the cell gel preparation; and a method for reducing the shear damage on cells in a shear environment using the nucleic acid hydrogel.

The cell gel preparation of the present invention can reduce the shear damage on cells caused by the shear environment including the injection process and the friction in the joint, and the cells in the cell gel preparation are hardly damaged in the shear environment. Specifically, the nucleic acid hydrogel of the present invention provides anti-shear protection for cells during the cell injection process, and the cell survival rate is increased from 85% to 99% or more compared with that achieved by the traditional injection method; the nucleic acid hydrogel provides anti-shear protection for cells when simulating the shear environment in the joint, and the cell survival rate is increased from 75% to 99% or more compared with that in the culture medium; and the effect of the cells delivered by the nucleic acid hydrogel in the treatment of knee osteoarthritis in rabbits is significantly better than that of the traditional cell therapy.

### BRIEF DESCRIPTION OF THE DRAWINGS

FIG. 1 shows result graphs of testing rheological properties (including time scan and strain sweep) of a cell-free hydrogel and a nucleic acid hydrogel loaded with mesenchymal stem cells.

FIG. 2 shows process diagrams of injecting cells through the traditional injection method and the nucleic acid hydrogel and the survival rate of cells determined by labeling them via Calcein-AM/PI staining before and after injection through both injection methods.

FIG. 3 shows statistical graphs of the survival rate of cells before and after injection through both injection methods.

FIG. 4 shows process diagrams of simulating the shear environment in the joint and the survival rate of cells determined by labeling them via Calcein-AM/PI staining before and after the cells are subjected to friction in the culture medium and in the hydrogel.

FIG. 5 shows a statistical graph of the survival rate of cells in the culture medium and in the hydrogel before and after being subjected to friction when simulating the shear environment in the joint.

FIG. 6 shows general views of the knee joint 12 weeks and 24 weeks after the treatment of knee osteoarthritis in rabbits with the nucleic acid hydrogel loaded with mesenchymal stem cells administrated by intra-knee joint injection.

FIG. 7 shows staining images of the knee joint tissue 12 weeks and 24 weeks after the treatment of knee osteoarthritis in rabbits with the nucleic acid hydrogel loaded with mesenchymal stem cells administrated by intra-knee joint injection.

FIG. 8 shows the effect of different nucleic acid solid contents on the survival rate of cells after injection.

### DETAILED DESCRIPTION OF EMBODIMENTS

The technical terms mentioned in this specification have the same meanings as those generally understood by those skilled in the art, and in case of conflict, the definitions in this specification will control.

In a first aspect, the present invention relates to a cell gel preparation for treating and/or preventing acute or chronic osteoarthritis and/or symptoms thereof by means of intra-articular injection, comprising a nucleic acid hydrogel loaded with mesenchymal stem cells.

In an embodiment, the nucleic acid hydrogel comprises: a scaffold unit comprising a scaffold core and at least three single-stranded nucleic acids bound to the scaffold core, each single-stranded nucleic acid having at least one scaffold cohesive end; a cross-linking unit comprising a cross-linking core and at least two single-stranded nucleic acids bound to the cross-linking core, each single-stranded nucleic acid having at least one cross-linking cohesive end; and an aqueous medium; wherein the scaffold unit and the cross-linking unit form a three-dimensional spatial network structure by cross-linking the scaffold cohesive ends and the cross-linking cohesive ends in a complementary base-pairing manner.

In an embodiment, the nucleic acid comprises deoxyribonucleic acid (DNA), ribonucleic acid (RNA), peptide nucleic acid (PNA), locked nucleic acid (LNA), and the like, preferably deoxyribonucleic acid (i.e., DNA). The nucleic acid may be a natural D-nucleic acid or L-nucleic acid, and the L-nucleic acid refers to a nucleic acid polymerized from L-nucleotides. Specifically, L-DNA refers to DNA polymerized from L-deoxyribonucleotides. The nucleic acid is preferably an L-nucleic acid.

In an embodiment, the scaffold unit comprises a scaffold core and at least three single-stranded nucleic acids bound to the scaffold core, each single-stranded nucleic acid having at least one scaffold cohesive end. Preferably, the scaffold core is a nucleic acid, in particular it may be a D-nucleic acid or an L-nucleic acid, and more in particular it may be a D-DNA or an L-DNA. In an embodiment, the nucleic acid as the scaffold core has a complementary pairing region, which may be 4-150 bp, preferably 5-50 bp, more preferably 6-30 bp, and more preferably 8-20 bp in length.

In an embodiment, the scaffold core may be a polypeptide, which is a compound of two or more amino acids linked together by peptide bonds. The polypeptide as the scaffold core specifically encompasses dipeptides, tripeptides, tetrapeptides, and the like. In addition, the polypeptide of the present invention also encompasses oligopeptides, proteins, proteins, and the like.

In an embodiment, the cross-linking unit comprises a cross-linking core and at least two single-stranded L-nucleic acids bound to the cross-linking core, each single-stranded L-nucleic acid having at least one cross-linking cohesive end. Preferably, the cross-linking core may be a nucleic acid, in particular it may be a D-nucleic acid or an L-nucleic acid, and more in particular it may be a D-DNA or an L-DNA. In an embodiment, the nucleic acid as the cross-linking core has a complementary pairing region, which may be 4-150 bp, preferably 5-50 bp, more preferably 6-30 bp, and more preferably 8-20 bp in length.

In an embodiment, the scaffold cohesive end or the cross-linking cohesive end is 4 nt or more in length, which facilitates its stable cross-linked state under physiological conditions. Preferably, the scaffold cohesive end or the cross-linking cohesive end is 150 nt or less, preferably 50 nt or less, more preferably 30 nt or less, more preferably 30-50 nt, and more preferably 20 nt or less in length.

In an embodiment, when the scaffold core and the cross-linking core are the same, and the single-stranded nucleic acids bound to the scaffold core and the single-stranded nucleic acids bound to the cross-linking core are the same, for example, the same L-nucleic acid; and the number of the single-stranded nucleic acids bound to the scaffold core and the number of the single-stranded nucleic acids bound to the cross-linking core (both≥3), the scaffold unit and the cross-linking unit are the same. Thus, in an embodiment, the scaffold unit and the cross-linking unit are the same. In an embodiment, the scaffold unit and the cross-linking unit are different.

In an embodiment, the molar ratio of the scaffold unit to the cross-linking unit in the nucleic acid hydrogel is 2:1-1:3, preferably 1:1-1:2, and more preferably 1:1.5.

In an embodiment, the scaffold unit and the cross-linking unit form a three-dimensional spatial network structure by cross-linking the scaffold cohesive ends and the cross-linking cohesive ends in a complementary base-pairing manner. Preferably, the scaffold unit, the cross-linking unit and the three-dimensional spatial network structure are in a stable cross-linked state under physiological conditions (37° C., pH 7.2-7.4, 0.9 wt % NaCl, isotonic).

In an embodiment, the aqueous medium refers to water or an aqueous solution. As the aqueous solution, a buffer containing a buffer salt is preferred. The aqueous solution is preferably capable of forming an environment similar to the in vivo microenvironment of the stem cells, such as physiological conditions (37° C., pH 7.2-7.4, 0.9 wt % NaCl, isotonic).

In an embodiment, the nucleic acid hydrogel has a suitable mechanical strength, for example, the mechanical strength may be 0.1 Pa or more, preferably 1 Pa or more, more preferably 10 Pa or more, preferably 10000 Pa or less, and more preferably 1000 Pa or less.

In an embodiment, the nucleic acid hydrogel has a predominant storage modulus G′ relative to the loss modulus G″ at all strain values from 0.1 to 100 rad/s as measured by the sweep-frequency measurement method at 25° C. and at a strain amplitude of 1% strain. Preferably, the ratio of the storage modulus G′ to the loss modulus G″ is greater than 2, and more preferably greater than 10 at strain values of 0.1 to 100 rad/s.

In an embodiment, the hydrogel of the present invention may have a desirable stability, for example, it may maintain its structure stably in the presence of a restriction endonuclease for 24 hours, preferably 36 hours, preferably 48 hours, or more.

In an embodiment, the scaffold unit or the cross-linking unit of the nucleic acid hydrogel may comprise a CpG sequence. CpG sequence is a palindromic sequence using cytosine-phosphate-guanosine (CpG) dinucleotides as the core, with two purines at the 5′ end, and two pyrimidines at the 3′ end, that is, 5′-PurPur-CG-PyrPyr-3′. The CpG sequence can be recognized by mammalian cells to trigger a series of body defense mechanisms, including complement activation, phagocytosis, expression of pro-inflammatory cytokine genes, etc. Currently, CpG sequences known to have a strong immunostimulatory effect include, for example, 5′-TCCATGACGTTCCTGACGTT-3′, and the like.

In an embodiment, the sequence of the scaffold unit is 5′-CGATTGACTCTC CACGCTGTCCTAACCATGACCGTCGAAG-3′, 5′-CGATTGACTCTCCTTCGACG GTCATGTACTAGATCAGAGG-3′, or 5′-CGATTGACTCTCCCTCTGATCTAGTAG TTAGGACAGCGTG-3′. In an embodiment, the sequence of the cross-linking unit is 5′-GAGAGTCAATCGTCTATTCGCATGAGAATTCCATTCACCGTAAG-3′, or 5′-GAGAGTCAATCGCTTACGGTGAATGGAATTCTCATGCGAATAGA-3′.

The nucleic acid hydrogel of the present invention can be prepared by the method described in CN201610740754X. For example, DNA single strands (i.e., the scaffold unit and the cross-linking unit) can be prepared separately, and then mixed to obtain the hydrogel of the present invention through self-assembly. Alternatively, the scaffold unit and the cross-linking unit can be mixed with the aqueous medium separately to obtain a solution of the scaffold unit in the aqueous medium and a solution of the cross-linking unit in the aqueous medium, and then the two solutions are mixed to cross-link to form a three-dimensional spatial network structure to obtain the hydrogel of the present invention.

In an embodiment, the mesenchymal stem cells are embedded or dispersed in the nucleic acid hydrogel; and preferably, the mesenchymal stem cells are embedded in the nucleic acid hydrogel.

In an embodiment, the mesenchymal stem cells are from bone marrow, umbilical cord or placenta; and the mesenchymal stem cells are derived from any one of bone marrow mesenchymal stem cells, adipose mesenchymal stem cells, peripheral blood mesenchymal stem cells, synovial mesenchymal stem cells, umbilical cord mesenchymal stem cells, and umbilical cord blood mesenchymal stem cells. The mesenchymal stem cells have desirable anti-inflammatory and regenerative properties.

In an embodiment, the mesenchymal stem cells have a density of 10⁵ to 10⁸ cells/mL (per milliliter of cell gel preparation), preferably 2×10⁷ to 5×10⁷ cells/mL, and more preferably 1×10⁷ cells/mL.

In an embodiment, the gel has a nucleic acid solid content of 0.7-5.0% (mass/volume ratio, nucleic acid solid content per milliliter of gel) to protect the cells during the injection process. When the nucleic acid solid content is less than 0.7% (mass percentage), the gel cannot be formed; and when the nucleic acid content in the gel is greater than 5.0%, the injection is difficult due to its high strength and excessive resistance during the injection process. In a preferred embodiment, the nucleic acid solid content is 1.5-4.0%, 2.0-4.0%, 3.0-4.0%, 3.5-4.0%, or 3.8%.

In an embodiment, the mesenchymal stem cells are added or mixed into the hydrogel after the nucleic acid hydrogel is formed, thereby forming the cell gel preparation. In an embodiment, the mesenchymal stem cells are mixed with the DNA assembly to form the nucleic acid hydrogel loaded with the mesenchymal stem cells according to a conventional method. In an embodiment, the mesenchymal stem cells are mixed with DNA single strands (3 scaffold units and 2 cross-linking units) to form a cell-loaded DNA assembly and then the nucleic acid hydrogel.

In an embodiment, the cell gel preparation further comprises an anti-inflammatory agent, and the anti-inflammatory agent is preferably selected from steroidal anti-inflammatory compounds (e.g., prednisolone, dexamethasone, betamethasone and triamcinolone), non-steroidal anti-inflammatory compounds (e.g., ibuprofen, diclofenac, naproxen, etc.), anti-rheumatic drugs (e.g., methotrexate, leflunomide, etc.), anti-CD20 agents, anti-cytokine agents (e.g., anti-IL1, anti-IL6 and anti-IL-17), anti-TNF agents (e.g., infliximab, etanercept, adalimumab, rituximab, etc.), or a mixture thereof.

In a second aspect, the present invention relates to a method for treating or preventing acute and chronic osteoarthrosis and associated inflammation-derived symptoms (especially osteoarticular pain and activity or function loss) using the cell gel preparation. In an embodiment, the disease or symptoms thereof is selected from, for example, osteoarthritis, degenerative arthritis, knee joint disease, degenerative joint disease of the hip, and other inflammatory general conditions involving joints, for example autoimmune diseases (especially rheumatoid arthritis and systemic lupus erythematosus (SLE)), spondyloarthropathy, polymyalgia rheumatica, ankylosing spondylitis, Reiter's syndrome, psoriatic arthropathy, enteropathic arthritis (associated with inflammatory bowel diseases such as hemorrhagic colitis and Crohn's disease), neuropathic arthropathy, acute rheumatic fever, gout, chondrocalcinosis, hydroxyapatite crystal deposition disease, Lyme disease and all other degenerative joint diseases.

In a third aspect, the present invention relates to a method for reducing the shear damage on cells in a shear environment using the nucleic acid hydrogel, comprising: (i) providing the nucleic acid hydrogel comprising the scaffold unit and the cross-linking unit, and (ii) loading the nucleic acid hydrogel with mesenchymal stem cells; or (i) separately preparing the scaffold unit and the cross-linking unit of the nucleic acid hydrogel, and (ii) mixing mesenchymal stem cells with the scaffold unit and the cross-linking unit to obtain the cell gel preparation of the present invention through self-assembly; or determining the anti-shear protective effect of the cell-loaded nucleic acid hydrogel on cells by a cell injection test or the like.

In a fourth aspect, the present invention relates to the use of the cell gel preparation in the manufacture of a medicament for the treatment or prevention of acute and chronic osteoarthrosis and associated inflammation-derived symptoms.

In an embodiment, the nucleic acid hydrogel is as described above. In an embodiment, the mesenchymal stem cells are embedded or dispersed in the nucleic acid hydrogel. In an embodiment, the shear environment comprises injection process and intra-articular internal friction.

### EXAMPLES

### Experimental Materials

The nucleic acid hydrogel was prepared using the method disclosed in CN201610740754X. The DNA single strands (Y1, Y2 and Y3) and (L1C and L2C) shown in Table 1 were first self-assembled to DNA assemblies (which were 1 mmol/L of scaffold unit and 1.5 mmol/L of cross-linking unit) in the aqueous medium, respectively, and the DNA assemblies were then mixed to form the desired DNA hydrogel sample (i.e., the nucleic acid hydrogel).

TABLE 1
Sequence information of DNA used to construct DNA hydrogel

| Name | nt | ϵ | M | Sequence |
|---|---|---|---|---|
| Y1 | 40 | 370600 | 12177 | 5′-CGATTGACTCTCCACGCTGTCCTAACCATGACCGTCGAAG-3′ |
| Y2 | 40 | 383000 | 12287 | 5′-CGATTGACTCTCCTTCGACGGTCATGTACTAGATCAGAGG-3′ |
| Y3 | 40 | 378300 | 12278 | 5′-CGATTGACTCTCCCTCTGATCTAGTAGTTAGGACAGCGTG-3′ |
| L1C | 44 | 433300 | 13515 | 5′-GAGAGTCAATCGTCTATTCGCATGAGAATTCCATTCACCGTAAG-3′ |
| L2C | 44 | 444200 | 13644 | 5′-GAGAGTCAATCGCTTACGGTGAATGGAATTCTCATGCGAATAGA-3′ |

ϵ denotes the absorption coefficient in L/mol.cm; the sequences Y1, Y2 and Y3 form the Y-scaffold unit; and the sequences L1C and L2C form the cross-linking unit.

The cell culture medium was Minimum Essential Medium (MEM) alpha (Gibco, Thermo Fisher, USA).

The Live-Dead Cell Staining Kit was Calcein-AM/PI (Solarbio, Beijing).

The rheological properties were tested using the Komexus rheometer (Malvern); and the cell images were acquired using the LSM 710Meta laser scanning confocal microscope (Carl Zeiss AG, Germany).

### Example 1: Preparation Of Nucleic Acid Hydrogel Loaded With Mesenchymal Stem Cells

1 mmol/L of Y-scaffold unit in Table 1 above was fully dissolved in a certain volume of suspension of stem cells (derived from bone marrow mesenchymal stem cells of rabbits, extracted in laboratory), and 1.5 mmol/L of cross-linking unit in Table 1 above was fully dissolved in an equal volume of solution, and the two resultants were mixed to form the gels after a few seconds. The resulting nucleic acid hydrogel had a final cell concentration of 1×10⁷ cells/ml and a nucleic acid solid content of 3.8% (mass percentage).

### Example 2: Rheological Testing Of Cell-Loaded Nucleic Acid Hydrogel

The nucleic acid hydrogel loaded with mesenchymal stem cells prepared in Example 1 was taken out with a medicine spoon, and placed on the test board of the rheometer. The tapered plate of the rheometer was adjusted to slowly descend to contact the hydrogel samples, and finally, the distance between the tapered plate and the board was fixed at 150 μm. This section involved two types of rheological tests in total.

The first was the time scan test, that is, the samples were subjected to continuous shear treatment in a certain period of time while keeping the scanning frequency unchanged, the ability of the samples to resist shear was tested, and the mechanical strength of different samples was further reflected by comparing the ratio of the storage modulus to the loss modulus. The parameters set in this experiment were as follows: strain=1%, frequency=1 Hz, and constant temperature at 25° C.

The second was the strain sweep test, which reflected that the samples exhibited shear thinning behaviors by measuring the change trend of the storage modulus and the loss modulus of different samples under different strains. The parameters set in this experiment were as follows: strain range=0.1 to 1000%, frequency=1 Hz, and constant temperature at 25° C.

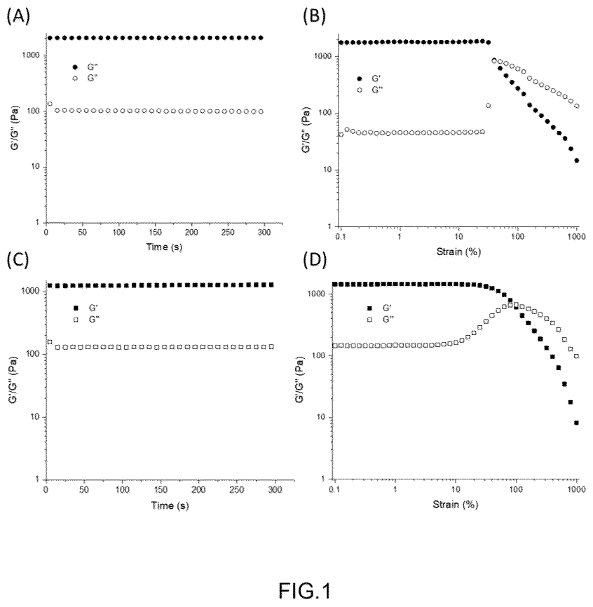

FIG. 1 and FIG. 1B show the results of the rheological test of a cell-free hydrogel in the time mode and the strain mode, and FIG. 1C and FIG. 1D show the results of the rheological test of a cell-containing hydrogel in the time mode and the strain mode. It can be seen from FIG. 1 that the nucleic acid hydrogel loaded with mesenchymal stem cells still has good mechanical strength to support cells, while still having shear thinning properties.

### Example 3: Shear Protection Effect Of Cell-Loaded Nucleic Acid Hydro2E1 On Cells

This example investigates the anti-shear protective effect of the nucleic acid hydrogel prepared in Example 1 on cells during the cell injection process and when simulating the shear environment in the joint.

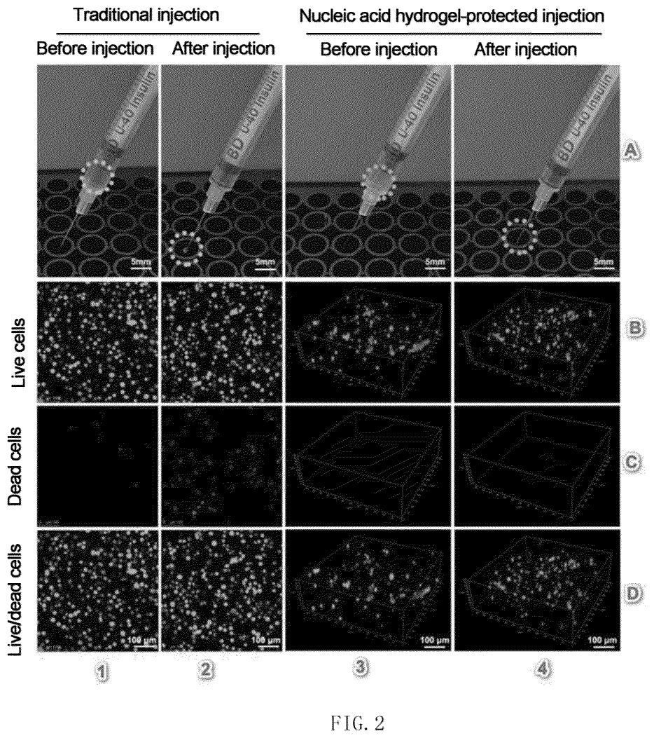

Cells were injected via the cell-loaded nucleic acid hydrogel of Example 1 using a 29 G insulin syringe (in which the needle diameter was only 330 μm), and the cells in the culture medium were used as the control. The cell survival rate was characterized by Live/Dead staining after injection. The experimental results are as shown in FIG. 2 and FIG. 3. FIG. 2 shows process diagrams of injecting cells through the traditional injection method and the nucleic acid hydrogel and the survival rate of cells determined by labeling them via Calcein-AM/PI staining before and after injection through both injection methods. In FIG. 2, row A shows the injection method, row B shows the distribution of live cells (live cells are green bright spots (in the original image) or white spots (in the black and white image)), row C shows the distribution of dead cells (dead cells are red bright spots (in the original image) or light gray spots (in the black and white image)), and row D shows the distribution of live/dead cells. For convenience, row C lists the distribution of dead cells separately. It can be seen from row C in FIG. 2 that before injection through the traditional injection method, many dead cells were found, such as area 1C in FIG. 2, whereas before nucleic acid hydrogel-protected injection, no dead cells were found, such as area 3C in FIG. 2; and after injection, the traditional injection method resulted in a large number of cell deaths, such as area 2C in FIG. 2, whereas after nucleic acid hydrogel-protected injection, almost no cell death was found, such as area 4C in FIG. 2. This indicates that the nucleic acid hydrogel loaded with mesenchymal stem cells can provide protection for cells (i.e., protection before injection), and the protective effect of the nucleic acid hydrogel loaded with mesenchymal stem cells on cells after injection is particularly obvious.

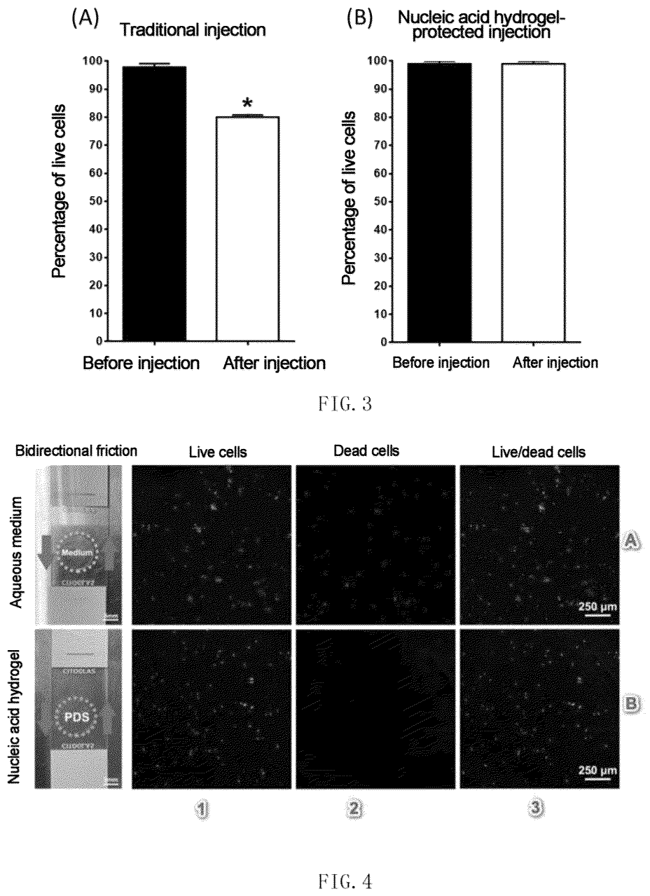

In order to simulate the shear environment in the joint, the nucleic acid hydrogel loaded with mesenchymal stem cells of Example 1 was placed between two cover slips with a distance of 330 μm, and the shear environment in the joint was simulated by bidirectional sliding of the two cover slips. The cell survival rate was characterized by Live/Dead staining after bidirectional sliding for 2 min. The experimental results are as shown in FIG. 4 and FIG. 5. FIG. 4 shows process diagrams of simulating the shear environment in the joint and the survival rate of cells determined by labeling them via Calcein-AM/PI staining before and after the cells were subjected to friction in the aqueous medium and in the hydrogel. Friction in the aqueous medium resulted in substantial cell death, such as area 2A in FIG. 4 (dead cells are red bright spots (in the original image) or light gray spots (in the black and white image)), whereas almost no dead cells were observed with the nucleic acid hydrogel.

It can be seen from the results of these two experiments that compared with the direct injection of a cell suspension, the nucleic acid hydrogel can provide anti-shear protection for cells during the injection process and when simulating the shear environment in the joint, and it is almost difficult to observe the production of dead cells, which greatly improves the survival rate of cells in the shear environment. Specifically, the nucleic acid hydrogel provided anti-shear protection for cells during the cell injection process, and the cell survival rate was increased from 85% to 99% or more compared with that achieved by the traditional injection method; and the nucleic acid hydrogel provided anti-shear protection for cells when simulating the shear environment in the joint, and the cell survival rate was increased from 75% to 99% or more compared with that in the culture medium.

### Example 4: Treatment Of Knee Osteoarthritis In Rabbits With Nucleic Acid Hydrogel Loaded With Mesenchymal Stem Cells Administrated By Intra-Knee Joint Injection

Skeletally mature male New Zealand rabbits (age: 6 months old, weight: 3.5±0.5 Kg, n=60) were purchased from the Animal Experiment Center of Peking University. Osteoarthritis was induced in the rabbits by anterior cruciate ligament transection plus medial meniscectomy. All animal experimental procedures were approved by the Animal Care and Use Committee of Peking University, and were in accordance with the Guidelines for the Care and Use of Laboratory Animals (National Academy of Sciences Press, published by the National Institutes of Health, No. 85-23, revised in 1996). The specific steps were as follows. After anesthesia and routine aseptic preparation, the joint cavity was entered through the medial patellar incision, the anterior cruciate ligament was subjected to transection, and the medial meniscus was completely resected. The loss of stability of the knee joint was confirmed by the anterior drawer test. After the operation, all animals were put back into the corresponding rabbit cages, allowed to move freely, given necessary infection prevention and pain relief treatment, and subjected to knee functional recovery.

All animals were randomly divided into five groups, and separately subjected to left knee surgery and treatment intervention through injection , including 1, Sham group: the skin was simply incised and then sutured; 2, Blank group: the knee osteoarthritis modeling surgery was simply performed in the rabbits; 3, PDS group: the knee osteoarthritis in the rabbits was treated with the nucleic acid hydrogel alone through injection; 4, MSCs group: the knee osteoarthritis in the rabbits was treated with mesenchymal stem cells alone through injection; and 5, Hybrid group: the knee osteoarthritis in the rabbits was treated with the nucleic acid hydrogel loaded with mesenchymal stem cells through injection. All injections were carried out at Week 8 after the osteoarthritis modeling surgery in the rabbits, once a week, for a total of 3 times. 200 μl of nucleic acid hydrogel per knee was injected to the PDS group at each time; 200 μl of cell suspension in basal medium per knee was injected to the MSCs group at each time, with a cell concentration of 1×10⁷ cells/ml; and 200 μl of nucleic acid hydrogel loaded with mesenchymal stem cells of Example 1 per knee was injected to the Hybrid group at each time, with a cell concentration of 1×10⁷ cells/ml. At Week 12 and Week 24 after the completion of the treatment through injection, the rabbits in each group were euthanized, and knee joint samples were collected for further study.

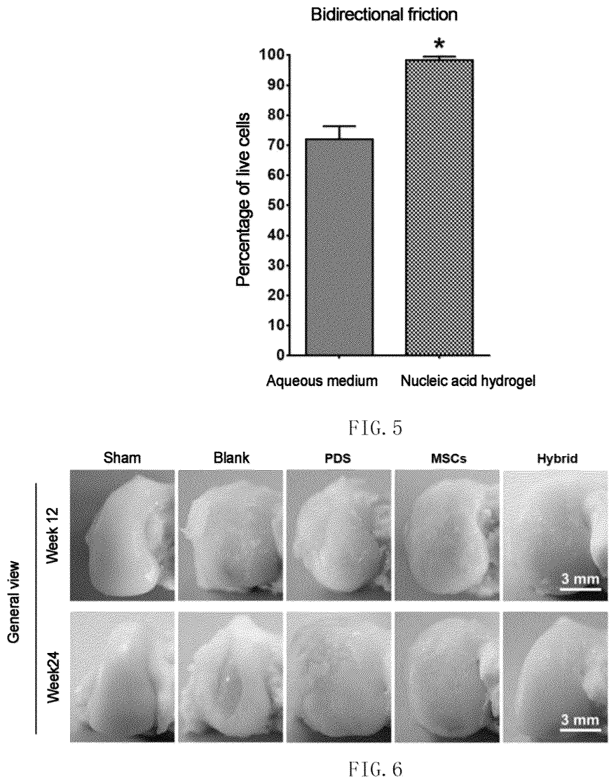

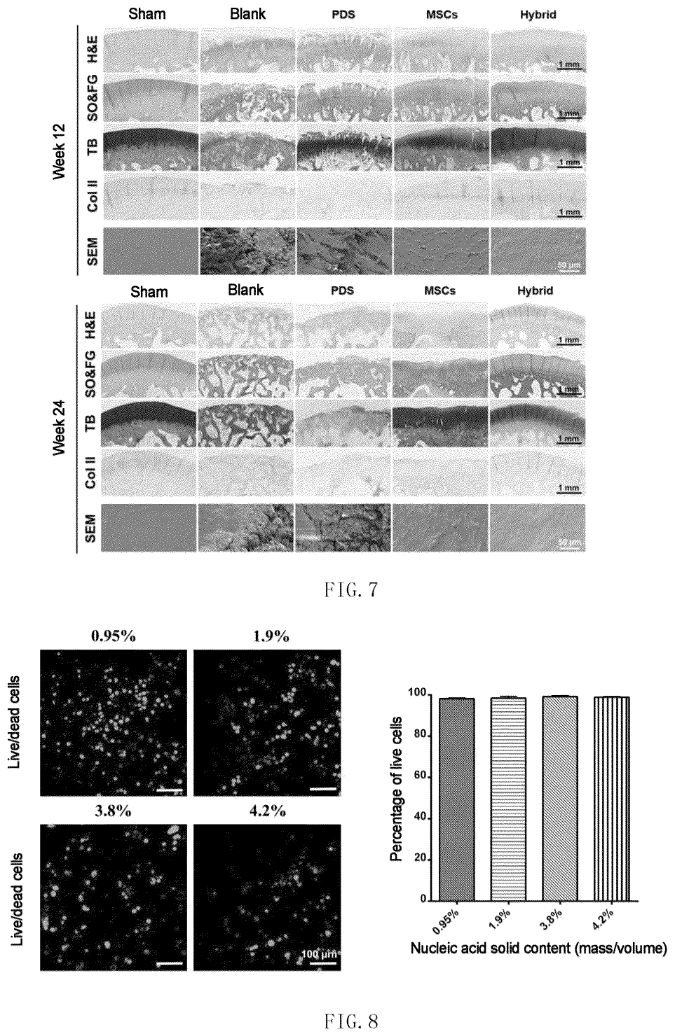

It can be seen from the general observation results that the Blank group and the PDS group showed cartilage erosion and cartilage defect progressively aggravated over time, whereas the cartilage injury in the MSCs treatment group and the Hybrid treatment group was significantly improved, and the general observation result in the Hybrid group was closer to that in the normal cartilage (the Sham group). The sample tissues were further subjected to H&E, Safranine O-Fast Green, and toluidine blue staining, as well as type II collagen immunohistochemistry. Compared with the MSCs treatment group, the cartilage morphology and type II collagen expression in the nucleic acid hydrogel loaded with mesenchymal stem cells treatment group (the Hybrid group) were closer to those in the normal cartilage tissue, especially at Week 24. Similarly, it was found by observing the microstructure of the cartilage surface with a scanning electron microscope (SEM) that the cartilage surface in the traditional aqueous solution loaded with mesenchymal stem cells treatment group (the MSCs group) was not cracked, but still showed a large number of hills. However, the cartilage surface in the Hybrid group was smoother, with only tiny irregularities. The results are as shown in FIG. 6 and FIG. 7.

### Example 5: Effect Of Nucleic Acid Solid Content On Nucleic Acid Hydrogel Loaded With Mesenchymal Stem Cells

Mesenchymal stem cell-loaded nucleic acid hydrogels with a nucleic acid solid content of 0.95%, 1.9% and 4.2% were prepared in a similar manner to Example 1, and then these three nucleic acid hydrogels loaded with mesenchymal stem cells were tested in a similar manner to Example 3 for the anti-shear protective effect on cells during the cell injection process. 24 hours after injection, no dead cells were found with the mesenchymal stem cell-loaded nucleic acid hydrogels with a nucleic acid solid content of 0.95%, 1.9% and 4.2%, which was similar to the observation result with the mesenchymal stem cell-loaded nucleic acid hydrogel with a nucleic acid solid content of 3.8%. However, when prepared in the same manner, the mesenchymal stem cell-loaded nucleic acid hydrogel with a nucleic acid solid content of 3.8% provided more uniform cell distribution, more suitable nucleic acid strength, and favorable injection. The results are as shown in FIG. 8.

Although the examples of the present invention have been shown and described above, it will be understood that the above examples are exemplary and are not to be construed as limiting the present invention, and those of ordinary skill in the art can make changes, modifications, substitutions and variations to the above examples within the scope of the present invention.

1. A cell gel preparation, comprising a nucleic acid hydrogel loaded with mesenchymal stem cells.

2. The cell gel preparation of claim 1, wherein the nucleic acid hydrogel comprises: a scaffold unit comprising a scaffold core and at least three single-stranded nucleic acids bound to the scaffold core, each single-stranded nucleic acid having at least one scaffold cohesive end; a cross-linking unit comprising a cross-linking core and at least two single-stranded nucleic acids bound to the cross-linking core, each single-stranded nucleic acid having at least one cross-linking cohesive end; and an aqueous medium; wherein the scaffold unit and the cross-linking unit form a three-dimensional spatial network structure by cross-linking the scaffold cohesive ends and the cross-linking cohesive ends in a complementary base-pairing manner.

3. The cell gel preparation of claim 2, wherein the scaffold unit comprises a scaffold core and at least three single-stranded nucleic acids bound to the scaffold core, each single-stranded nucleic acid having at least one scaffold cohesive end; the scaffold core is a nucleic acid; and the nucleic acid as the scaffold core has a complementary pairing region, which is 4-150 bp in length.

4. The cell gel preparation of claim 2, wherein the cross-linking unit comprises a cross-linking core and at least two single-stranded L-nucleic acids bound to the cross-linking core, each single-stranded L-nucleic acid having at least one cross-linking cohesive end; the cross-linking core is a nucleic acid;
and the nucleic acid as the cross-linking core has a complementary pairing region, which is 4-150 bp in length.

5. The cell gel preparation of claim 2, wherein the molar ratio of the scaffold unit to the cross-linking unit in the nucleic acid hydrogel is 2:1-1:3.

6. The cell gel preparation of claim 2, wherein the scaffold unit, the cross-linking unit and the three-dimensional spatial network structure are in a stable cross-linked state under physiological conditions (37° C., pH 7.2-7.4, 0.9 wt % NaCl, isotonic).

7. The cell gel preparation of claim 2, wherein the nucleic acid hydrogel has a predominant storage modulus G′ relative to the loss modulus G″ at all strain values from 0.1 to 100 rad/s as measured by the sweep-frequency measurement method at 25° C. and at a strain amplitude of 1% strain.

8. The cell gel preparation of claim 2, wherein the scaffold unit or the cross-linking unit of the nucleic acid hydrogel comprises a CpG sequence.

9. The cell gel preparation of claim 1, wherein the mesenchymal stem cells are embedded or dispersed in the nucleic acid hydrogel.

10. The cell gel preparation of claim 1, wherein the mesenchymal stem cells have a density of 10⁵ to 10⁸ cells/mL (per milliliter of cell gel preparation).

11. The cell gel preparation of claim 1, wherein the cell gel preparation further comprises an anti-inflammatory agent.

12. A method of treating or preventing acute and chronic osteoarthrosis and associated inflammation-derived symptoms comprising administering the cell gel preparation of claim 1.

13. A method for reducing the shear damage on cells in a shear environment using the cell gel preparation of claim 1, comprising:
(i) providing the nucleic acid hydrogel comprising the scaffold unit and the cross-linking unit, and (ii) loading the nucleic acid hydrogel with mesenchymal stem cells; or (i) separately preparing the scaffold unit and the cross-linking unit of the nucleic acid hydrogel, and (ii) mixing mesenchymal stem cells with the scaffold unit and the cross-linking unit to obtain the nucleic acid hydrogel through self-assembly.

14. The method of claim 13, further comprising subjecting the cell gel preparation to a shear environment.

15. The method of claim 14, wherein the shear environment comprises injection process and intra-articular internal friction.

16. The method of claim 12, wherein the cell gel preparation is administered by intra-articular injection.

17. The cell gel preparation of claim 1, wherein the nucleic acid hydrogel has a predominant storage modulus G′ relative to the loss modulus G″ at all strain values from 0.1 to 100 rad/s as measured by the sweep-frequency measurement method at 25° C. and at a strain amplitude of 1% strain.

18. The cell gel preparation of claim 1, wherein the nucleic acid hydrogel comprises a CpG sequence.
